(19) United States
(12) Patent Application Publication
(10) Pub. No.: US 2019/0091078 A1
(43) Pub. Date: Mar. 28, 2019

(54) Diaper Accessory Attachment

(71) Applicant: Yaakov Friedman, Highland Mills, NY (US)

(72) Inventor: Yaakov Friedman, Highland Mills, NY (US)

(21) Appl. No.: 15/719,415

(22) Filed: Sep. 28, 2017

(51) Int. Cl. A61F 13/84; B65F 1/00; B08B 1/00; A47K 10/42; A61F 13/551

(52) U.S. Cl. CPC A61F 13/84; B65F 1/002; B65F 1/0026; B08B 1/006; A47K 2010/3266; A61F 13/8405; A61F 13/5519; B65F 2210/129; B65F 2240/132; A47K 10/421

(57) ABSTRACT

A diaper accessory is disclosed including an elongated sticker having an adhesive layer with baby wipes and a disposal bag provided thereto. The sticker includes a pair of envelopes useful for retaining baby wipes and a disposal bag. The adhesive layer includes a non-adhesive strip that is removable to exposed an adhesive component such that the elongated sticker is attachable to an outside of a diaper. The envelope containing the baby wipes is adhesively attachable to a front of the diaper, and the envelope containing the disposal bag is adhesively attachable to a rear of the diaper. Further, the baby diaper accessory of the present disclosure is scented to mask the unpleasant odor when changing a baby's diaper.

### BACKGROUND OF THE INVENTION

The following includes information that may be useful in understanding the present disclosure. It is not an admission that any of the information provided herein is prior art nor material to the presently described or claimed inventions, nor that any publication or document that is specifically or implicitly referenced is prior art.

### 1. Field of the Invention

The present invention relates generally to the field of infant accessories and more specifically relates to diaper attachment.

### 2. Description of the Invention

Disposable infant diapers are well known as are moistened towelettes and disposal bags. Under normal home conditions, these items are normally found in the home nursery and it is usually not a problem to change a soiled diaper, the normal required materials being a clean fresh diaper, a number of moist towels or disposable towelettes and a disposal container. However, when traveling with a child, transporting the above noted items in a changing bag or other type of container can become troublesome. A suitable solution is needed.

U.S. Pat. No. 4,931,052 to Ruth L. Feldman relates to a diaper with integral wiping cloth and disposal container. The described diaper with integral wiping cloth and disposal container includes a disposable diaper with an integral wiping cloth and disposal container forming an integrated baby changing system. The disposable diaper has a skin contacting moisture absorbing inner surface and a waterproof environment interfacing outer surface. A storage container means is mounted to the outer surface of the diaper and includes a liquid impermeable membrane formed so as to define a pocket for retaining a towelette therein in the moistened state until such time as the towelette is needed for wiping the skin. The pocket is expandable to form a disposal container for the soiled diaper and the spent towelette. A moist towelette is adapted to be stored within the storage container means.

### BRIEF SUMMARY OF THE INVENTION

In view of the foregoing disadvantages inherent in the known infant accessories art, the present disclosure provides a novel diaper accessory attachment. The general purpose of the present disclosure, which will be described subsequently in greater detail, is to provide a diaper accessory comprising an elongated sticker having an adhesive layer with baby wipes and a disposal bag included within. The sticker may include a pair of envelopes useful for retaining baby wipes and a disposal bag. Preferably, the envelope containing the baby wipes is adhesively attachable to a front of the diaper, and the envelope containing the disposal bag is adhesively attachable to a rear of the diaper. Further, the baby diaper accessory of the present disclosure may be scented to mask the unpleasant odor of urine and bowel movements.

A diaper accessory attachment is disclosed herein. The diaper accessory attachment may include: a diaper accessory; at least one envelope having a front-side and a back-side and configured to substantially enclose the diaper accessory; and an adhesive layer fixed to the back-side of the at least one envelope and configured to attach the at least one envelope to the diaper. Further, the at least one envelope may include a diaper accessory egress configured to permit extraction of the diaper accessory from the at least one envelope.

A method of using the diaper accessory attachment is also disclosed herein. The method of using diaper accessory attachment may comprise the steps of: providing the diaper accessory attachment for use with a diaper; removing a non-adhesive strip from an adhesive layer; positioning the adhesive layer such that a first-envelope may be adjacent a human anterior pelvic region of the diaper and a second-envelope may be adjacent a human posterior pelvic region of the diaper; attaching the adhesive layer to the diaper; pulling a pull-tab of the first-envelope thereby revealing at least one baby wipe; cleaning a private region of an infant via the at least one baby wipe; removing a disposal bag from a slit integrated with the second-envelope; opening the disposal bag; inserting the diaper that may be dirty into the disposal bag; tying a pair of handles of the disposal bag together thereby closing the disposal bag; and disposing the disposal bag containing the diaper that may be dirty along with any soiled baby wipes used to clean the infant.

For purposes of summarizing the invention, certain aspects, advantages, and novel features of the invention have been described herein. It is to be understood that not necessarily all such advantages may be achieved in accordance with any one particular embodiment of the invention. Thus, the invention may be embodied or carried out in a manner that achieves or optimizes one advantage or group of advantages as taught herein without necessarily achieving other advantages as may be taught or suggested herein. The features of the invention which are believed to be novel are particularly pointed out and distinctly claimed in the concluding portion of the specification. These and other features, aspects, and advantages of the present invention will become better understood with reference to the following drawings and detailed description.

### BRIEF DESCRIPTION OF THE DRAWINGS

The figures which accompany the written portion of this specification illustrate embodiments and methods of use for the present disclosure, diaper accessory attachment, constructed and operative according to the teachings of the present disclosure.

FIG. 1 is a perspective view of the diaper accessory attachment during an ‘in-use’ condition, according to an embodiment of the disclosure.

FIG. 2A is a front perspective view of an adhesive layer of the diaper accessory attachment of FIG. 1 comprising at least one envelope for storing a baby accessory, according to an embodiment of the present disclosure.

FIG. 2B is a rear perspective view of the adhesive layer of the diaper accessory attachment of FIG. 2A, according to an embodiment of the present disclosure.

FIG. 3 is a perspective view of the adhesive layer configured to attach to a diaper, according to an embodiment of the present disclosure.

FIG. 4 is a front perspective view of the adhesive layer comprising a first-envelope including a pull-tab and configured to retain baby wipes and a second-envelope comprising an integrated slit and configured to retain a disposal bag, according to an embodiment of the present disclosure.

FIG. 5 is a flow diagram illustrating a method of use for the diaper accessory attachment, according to an embodiment of the present disclosure.

The various embodiments of the present invention will hereinafter be described in conjunction with the appended drawings, wherein like designations denote like elements.

### DETAILED DESCRIPTION

As discussed above, embodiments of the present disclosure relate to infant accessories and more particularly to a diaper accessory attachment as used to improve the task of changing a dirty diaper.

Generally, the diaper accessory attachment may be useful for providing an attachable accessory to a diaper having both baby wipes and a disposal bag. The diaper accessory attachment may comprise an adhesive layer including a pair of envelopes useful for retaining baby wipes and a disposal bag respectively.

A first envelope containing the baby wipes may be adhesively attached to a front of the diaper and include a pull tab for unsealing and opening the envelope. A second envelope containing the disposal bag may attach to a back of the diaper. Preferably, the disposal bag may comprise a plastic bag having a pair of handles. The handles may be tied together. Additionally, the disposal bag may be fixedly coupled to an inside of the second envelope to prevent removal of the bag. In such a manner, once a soiled diaper has been removed from an infant, the disposal bag may be removed from a slit of the second envelope, opened, and then the dirty diaper may be pushed through the bottom of the bag such that the bag is reversed to envelope the diaper. In addition, the disposal bag may include a fragrance to mask the unpleasant odor of urine and bowel movements.

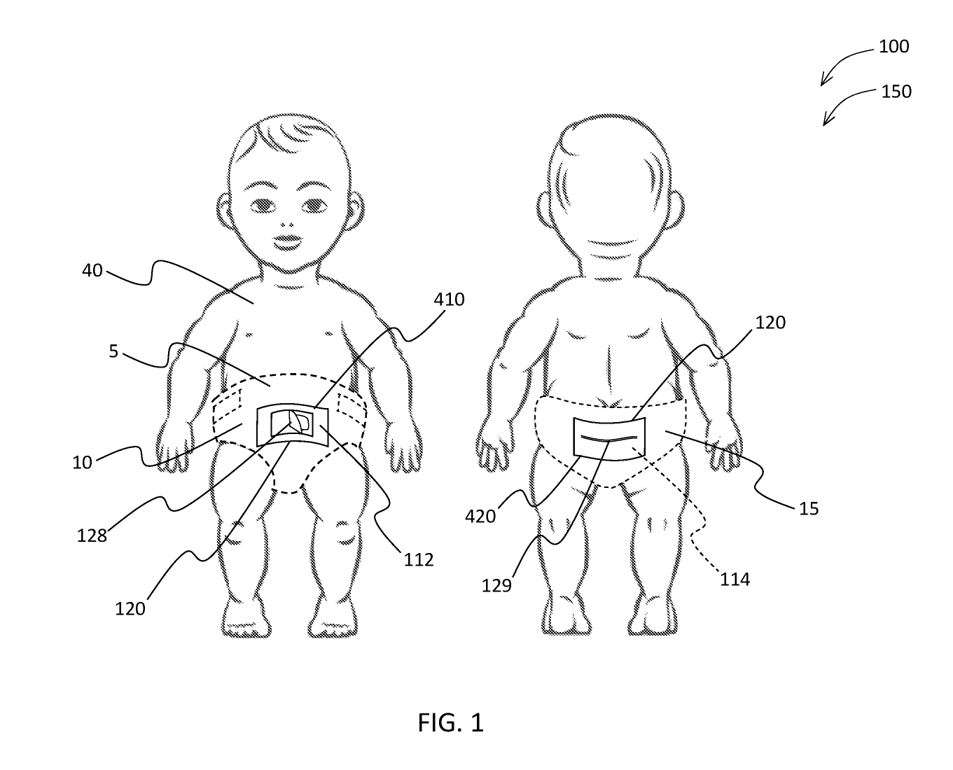

Referring now more specifically to the drawings by numerals of reference, there is shown in FIGS. 1-5, various views of a diaper accessory attachment 100. FIG. 1 shows a perspective view of a diaper accessory attachment 100 during an ‘in-use’ condition 150, according to an embodiment of the present disclosure. As illustrated, the diaper accessory attachment 100 for a diaper 5 may comprise: a diaper accessory 110; at least one envelope 120 having a front-side 122 and a back-side 124, the at least one envelope 120 configured to substantially enclose the diaper accessory 110, the at least one envelope 120 including a diaper accessory egress 126 configured to permit extraction of the diaper accessory 110 from the at least one envelope 120; and an adhesive layer 130 fixed to the back-side 124 of the at least one envelope 120, the adhesive layer 130 configured to attach the at least one envelope 120 to the diaper 5.

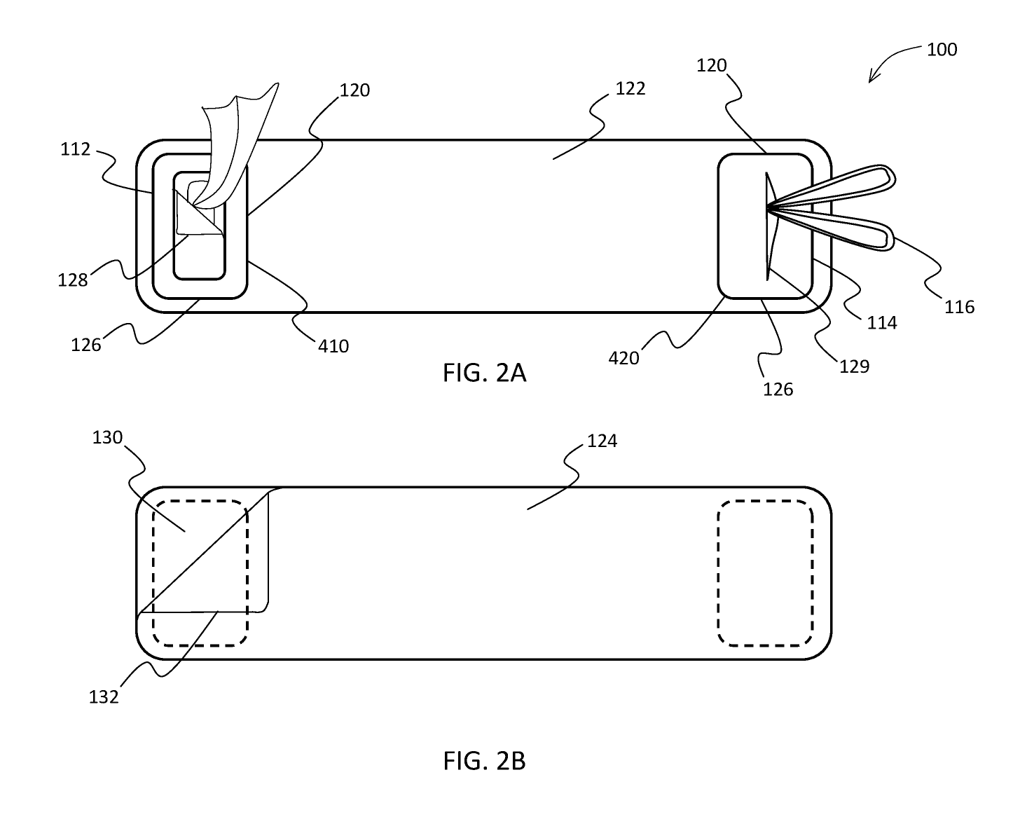

FIG. 2A shows a front perspective view of an adhesive layer 130 of the diaper accessory attachment 100 of FIG. 1 comprising at least one envelope 120 for storing a diaper accessory 110, according to an embodiment of the present disclosure. As above, the diaper accessory attachment 100 may include the diaper accessory 110, at least one envelope 120, and the adhesive layer 130 configured to adhere to an outside of the diaper 5.

Referring to FIG. 2A, the diaper accessory 110 may include at least one baby wipe 112. As may be appreciated by those familiar with the art, a baby wipe 112 essentially comprises a damp or moist toilette. It should be noted that the baby wipe 112 may include a sanitary agent. It should be further noted that the baby wipe 112 may comprise a scented agent having a pleasant fragrance. In one embodiment of the present invention, the at least one envelope 120 may house no more than six baby wipes 112.

In continuing to refer to FIG. 2A, the diaper accessory egress 126 of the at least one envelope 120 may include a pull tab 128. Preferably, the at least one envelope 120 that includes the pull tab 128 is configured to house a plurality of baby wipes 112. As should be appreciated, the pull tab 128 may act as a seal, the seal useful for retaining moisture of the plurality of baby wipes 120.

In continuing to refer to FIG. 2A, the diaper accessory 110 of the diaper accessory attachment 100 may include a diaper disposal bag 114 configured to enclose the diaper 5 after use. The diaper disposal bag 114 may include at least one handle 116 configured to carry the diaper disposal bag 114 with the diaper 5 inside after use and disposal of the diaper 5. Preferably, the diaper disposal bag 114 may include a pair of handles 116 configured to close the diaper disposal bag 114 with the diaper 5 inside after use of the diaper 5. The pair of handles 116 may be tied together to close the diaper disposal bag 114. The pair of handles 116 may further be useful for sanitarily carrying the diaper disposal bag 114 to a trash receptacle.

In one embodiment of the present invention, at least one envelope 120 of the diaper accessory attachment 100 may be scented. The scent may comprise a pleasant fragrance. The fragrance may be useful for masking unpleasant odors arising from the task of changing the diaper 5 that has been soiled. Further, it should be noted that the diaper accessory 110 (such as the disposal bag 114) may be scented in addition to, or in lieu of, the at least one envelope 120.

In still referring to FIG. 2A, it is shown that the diaper accessory egress 126 of at least one envelope 120 may be configured as a slit 129 which may be integrated with the at least one envelope 120. It should be noted that the at least one envelope 120 that includes the slit 129 may be configured to house the diaper disposal bag 114. In a preferred embodiment, the disposal bag 114 may be fixedly coupled to an interior surface of the at least one envelope 120 such that the disposal bag 114 is non-removable. In such a manner, once the diaper 5 that is soiled has been removed from a user 40, the disposal bag 114 may be removed from the slit 129, opened, and then the diaper 5 may be pushed through a bottom of the disposal bag 114 such that the disposal bag 114 is reversed to contain the diaper 5. As previously noted, the disposal bag 114 may include a fragrance to mask the unpleasant odor of urine and bowel movements.

Referring now to FIG. 2B showing a rear perspective view of the adhesive layer 130 of the diaper accessory attachment 100 of FIG. 2A, according to an embodiment of the present disclosure. As shown, at least one side of the adhesive layer 130 may include a non-adhesive strip 132. The non-adhesive strip 132 may be configured to peel from the at least one side of the adhesive layer 130 to expose an adhesive component or element of the adhesive layer 130.

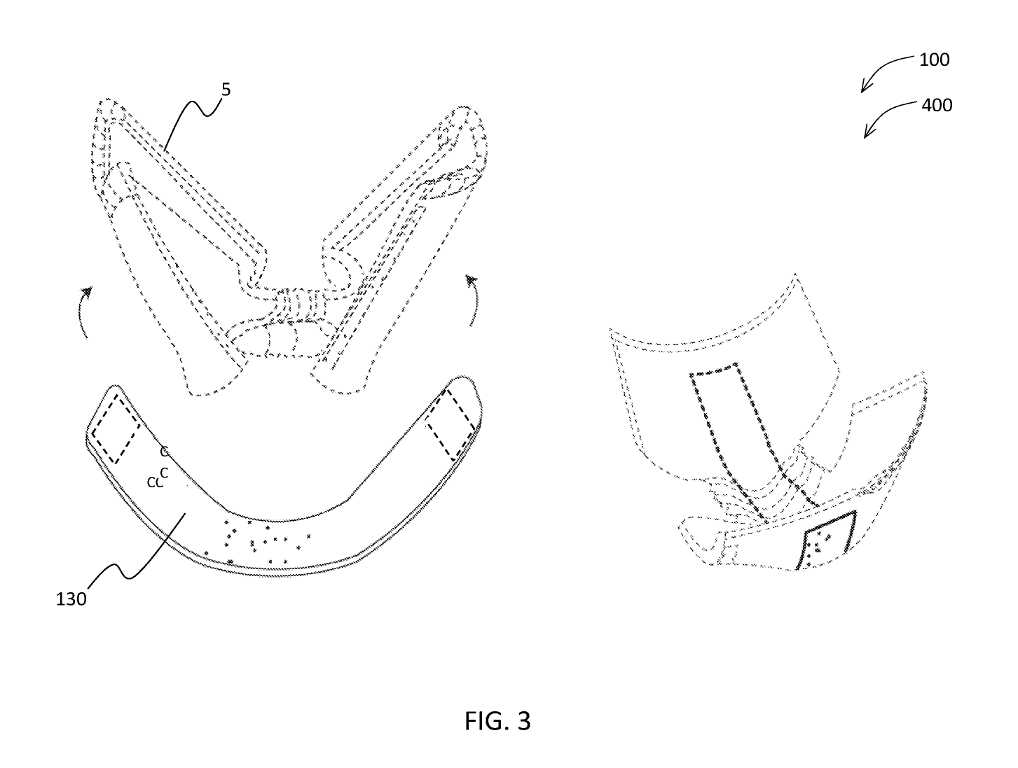

FIG. 3 shows a perspective view of the adhesive layer 130 configured to attach to a diaper 5, according to an embodiment of the present disclosure. As above, the adhesive layer 130 may include a non-adhesive strip 132. The non-adhesive strip 132 may be configured to peel from the at least one side of the adhesive layer 130 to expose an adhesive component or element of the adhesive layer 130. Once exposed, the adhesive element of the adhesive layer 130 may be adhesively coupled to an outside of the diaper 5. The adhesive element may be configured to allow the adhesive layer 130 to stick to the outside of the diaper 5 such that the adhesive layer 130 stays in place and is difficult to remove.

It should be noted that the front-side 122 of the at least one envelope 120 may be composed of fabric selected from a group consisting of cotton, hemp, bamboo, microfiber, and plastic. In such a manner, the front-side 122 of the at least one envelope preferably matches the same material as the diaper 5 such that the material is soft and non-abrasive.

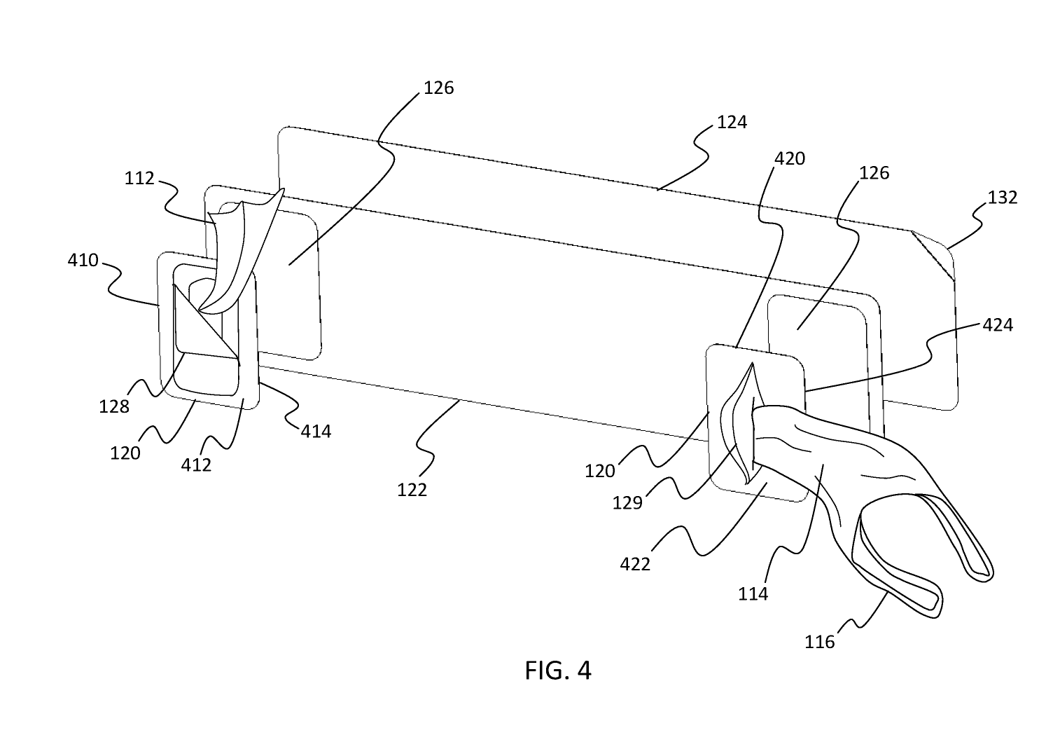

FIG. 4 shows a front perspective view of the adhesive layer 130 comprising a first-envelope 410 including the pull-tab 128 and configured to retain baby wipes 112 and a second-envelope 420 comprising an integrated slit 129 and configured to retain the disposal bag 114, according to a preferred embodiment of the present disclosure.

As shown, a baby wipe and disposal bag diaper accessory attachment for a diaper 400 may include: at least one baby wipe 112; a diaper disposal bag 114 including a pair of handles 116; a first-envelope 410 having a first-front-side 412 and a first-back-side 414, the first-envelope 410 configured to substantially enclose the at least one baby wipe 112, the first-envelope further including the pull tab 128 configured to permit extraction of at least one baby wipe 112 from the first-envelope 410; a second-envelope 420 having a second-front-side 422 and a second-back-side 424, the second-envelope 420 configured to substantially enclose the diaper disposal bag 114, the second-envelope 420 including a slit 129 integrated with the second-envelope 420, the slit 129 configured to permit extraction of the diaper disposal bag 114 from second-envelope 420; and the adhesive layer 130 fixed to the first-back-side 414 of the first-envelope 410 and the second-back-side 424 of the second-envelope 420, the adhesive layer 130 configured to attach the first-envelope 410 and the second-envelope 420 to the diaper 5, the adhesive layer further including the non-adhesive strip 132 configured to peel away from at least one side of the adhesive layer 130 to expose an adhesive component of the adhesive layer 130.

In continuing to refer to FIG. 4, it should be noted that the first-envelope 410 is configured for attachment to a human anterior pelvic region of the diaper 5 and the second-envelope 420 is configured for attachment to a human posterior pelvic region of the diaper 5. In such a manner, the first-envelope 410 containing the baby wipes 112 is conveniently accessible from a front of the diaper 5 when the diaper 5 is initially being changed, and the second-envelope 420 containing the disposal bag 114 is conveniently accessible from a back of the diaper 5 when the diaper 5 is ready for disposal.

According to one embodiment, the diaper accessory attachment 100 may be arranged as a kit. The kit may include the adhesive layer 130 having the first-envelope 410 containing baby wipes 112 and the second-envelope 120 containing the disposal bag 114. The kit may further include user instructions. The user instructions may detail functional relationships in relation to the structure of the diaper accessory attachment 100 (such that the diaper accessory attachment 100 can be used, maintained, or the like, in a preferred manner).

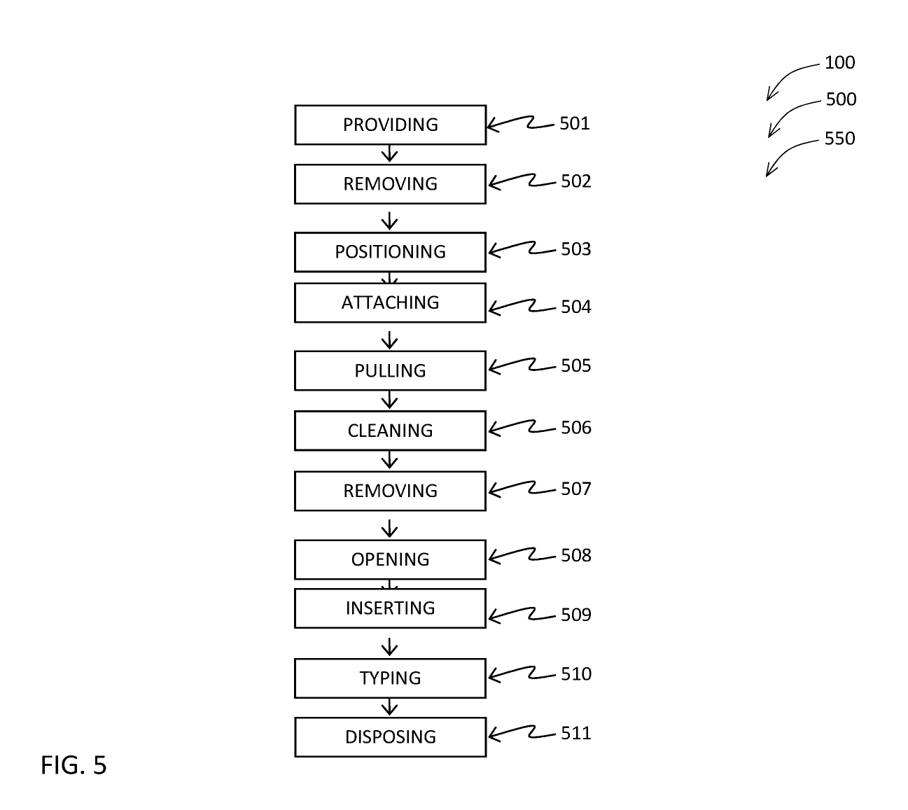

FIG. 5 is a flow diagram 550 illustrating a method of use 500 of the diaper accessory attachment 100, according to an embodiment of the present disclosure. As illustrated, the method of use 500 for the diaper accessory attachment 100 may include the steps of: step one 501, providing the diaper accessory attachment for use with a diaper; step two 502, removing a non-adhesive strip from an adhesive layer; step three 503, positioning the adhesive layer such that a first-envelope may be adjacent a human anterior pelvic region of the diaper and a second-envelope may be adjacent a human posterior pelvic region of the diaper; step four 504, attaching the adhesive layer to the diaper; step five 505, pulling a pull-tab of the first-envelope thereby revealing at least one baby wipe; cleaning a private region of an infant via the at least one baby wipe; step six 506, removing a disposal bag from a slit integrated with the second-envelope; step seven 507, opening the disposal bag; step eight 508, inserting the diaper that may be dirty into the disposal bag; step nine 509, tying a pair of handles of the disposal bag together thereby closing the disposal bag; and step ten 510, disposing the disposal bag containing the diaper that may be dirty and at least one baby wipe.

It should be noted that the steps described in the method of use can be carried out in many different orders according to user preference. The use of “step of” should not be interpreted as “step for”, in the claims herein and is not intended to invoke the provisions of 35 U.S.C. § 112(f). It should also be noted that, under appropriate circumstances, considering such issues as design preference, user preferences, marketing preferences, cost, structural requirements, available materials, technological advances, etc., other methods for diaper accessory attachment 100 (e.g., different step orders within above-mentioned list, elimination or addition of certain steps, including or excluding certain maintenance steps, etc.), are taught herein.

The embodiments of the invention described herein are exemplary and numerous modifications, variations and rearrangements can be readily envisioned to achieve substantially equivalent results, all of which are intended to be embraced within the spirit and scope of the invention. Further, the purpose of the foregoing abstract is to enable the U.S. Patent and Trademark Office and the public generally, and especially the scientist, engineers and practitioners in the art who are not familiar with patent or legal terms or phraseology, to determine quickly from a cursory inspection the nature and essence of the technical disclosure of the application.

1. A diaper accessory attachment for a diaper, the diaper accessory attachment comprising:
a diaper accessory;
at least one envelope having a front-side and a back-side, the at least one envelope configured to substantially enclose the diaper accessory, the at least one envelope including a diaper accessory egress configured to permit extraction of the diaper accessory from the at least one envelope; and
an adhesive layer fixed to the back-side of the at least one envelope, the adhesive layer configured to attach the at least one envelope to the diaper.

2. The diaper accessory attachment of claim 1, wherein the diaper accessory includes at least one baby wipe.

3. The diaper accessory attachment of claim 2, wherein the diaper accessory includes less than six baby wipes.

4. The diaper accessory attachment of claim 1, wherein the diaper accessory is a disposal bag configured to enclose the diaper after use.

5. The diaper accessory attachment of claim 4, wherein the disposal bag includes at least one handle configured to carry the disposal bag with the diaper inside after use of the diaper.

6. The diaper accessory attachment of claim 4, wherein the disposal bag includes a pair of handles configured to close the disposal bag with the diaper inside after use of the diaper.

7. The diaper accessory attachment of claim 1, wherein the diaper accessory is scented.

8. The diaper accessory attachment of claim 1, wherein the diaper accessory egress of the at least one envelope includes a pull tab.

9. The diaper accessory attachment of claim 8, wherein the at least one envelope that includes the pull tab is configured to house a plurality of baby wipes.

10. The diaper accessory attachment of claim 9, wherein the pull tab acts as a seal useful for retaining moisture of the plurality of baby wipes.

11. The diaper accessory attachment of claim 1, wherein the diaper accessory egress is configured as a slit integrated with the at least one envelope.

12. The diaper accessory attachment of claim 11, wherein the at least one envelope that includes the slit is configured to house a disposal bag.

13. The diaper accessory attachment of claim 12, wherein the disposal bag is fixedly coupled to an interior surface of the at least one envelope such that the disposal bag is non-removable.

14. The diaper accessory attachment of claim 1, wherein at least one side of the adhesive layer further includes a non-adhesive strip configured to separate from the at least one side to expose an adhesive component of the adhesive layer.

15. The diaper accessory attachment of claim 1, wherein the front-side of the at least one envelope is composed of fabric from a group consisting of cotton, hemp, bamboo, microfiber, and plastic.

16. A baby wipe and disposal bag diaper accessory attachment for a diaper, the baby wipe and disposal bag diaper accessory attachment comprising:
at least one baby wipe;
a diaper disposal bag including a pair of handles;
a first-envelope having a first-front-side and a first-back-side, the first-envelope configured to substantially enclose the at least one baby wipe, the first-envelope including a pull tab configured to permit extraction of at least one baby wipe from the first-envelope;
a second-envelope having a second-front-side and a second-back-side, the second-envelope configured to substantially enclose the diaper disposal bag, the second-envelope including a slit integrated with the second-envelope, the slit configured to permit extraction of the diaper disposal bag from second-envelope; and
an adhesive layer fixed to the first-back-side of the first-envelope and the second-back-side of the second-envelope, the adhesive layer configured to attach the first-envelope and the second-envelope to the diaper, the adhesive layer further including a non-adhesive strip configured to separate from at least one side of the adhesive layer to expose an adhesive component of the adhesive layer.

17. The baby wipe and disposal bag diaper accessory attachment of claim 16, wherein the first-envelope is configured for attachment to a human anterior pelvic region of the diaper and the second-envelope is configured for attachment to a human posterior pelvic region of the diaper.

18. The baby wipe and disposal bag diaper accessory attachment of claim 17, wherein the disposal bag is fixedly coupled to an interior surface of the second-envelope such that the disposal bag is non-removable.

19. The baby wipe and disposal bag diaper accessory attachment of claim 18, wherein the disposal bag and alternatively the second-envelope is scented.

20. A method of using a diaper accessory attachment, the method comprising the steps of:
providing the diaper accessory attachment for use with a diaper;
removing a non-adhesive strip from an adhesive layer;
positioning the adhesive layer such that a first-envelope is adjacent a human anterior pelvic region of the diaper and a second-envelope is adjacent a human posterior pelvic region of the diaper;
attaching the adhesive layer to the diaper;
pulling a pull-tab of the first-envelope thereby revealing at least one baby wipe;
cleaning a private region of an infant via the at least one baby wipe;
removing a disposal bag from a slit integrated with the second-envelope;
opening the disposal bag;
inserting the diaper that is dirty into the disposal bag;
tying a pair of handles of the disposal bag together thereby closing the disposal bag; and
disposing the disposal bag containing the diaper that is dirty and at least one baby wipe.
